(19) United States
(12) Patent Application Publication
(10) Pub. No.: US 2005/0015054 A1
(43) Pub. Date: Jan. 20, 2005

(54) Syringe needle protecting cover

(76) Inventor: Jen Chen Chen, Taipei Hsien (TW)

Correspondence Address: Yen Chen Chen, 235 Chung - Ho, Box 8-24, Taipei, TW

(21) Appl. No.: 10/672,191

(22) Filed: Sep. 24, 2003

(30) Foreign Application Priority Data
Jul. 16, 2003 (TW) 92212971

(51) Int. Cl. A61M 5/32

(52) U.S. Cl. 604/192

(57) ABSTRACT

A safety syringe needle protecting cover comprises a cover, a syringe tube and an elastic element. The cover has an outlet capable of being connected to a front end of a syringe cylinder. The cover receives a syringe needle. A syringe tube is a hollow tube for hiding the syringe needle therein. The syringe tube is coupled to the outlet of the cover; the syringe tube being movable in the outlet. The syringe needle is shielded by the syringe tube. An exposed end of the syringe tube has an injection end. The elastic element is an elastic element located along the axial direction of the syringe tube. Two end of the elastic element resisting against the outlet of the cover and the injection end of the syringe tube, respectively. By the elastic force of the elastic element, the syringe tube is pushed to the position hiding the syringe needle.

### FIELD OF THE INVENTION

The present invention relates to syringes, and particularly to a syringe heedle protecting cover, where when the syringe is used, the syringe needle protecting cover is unnecessary to be taken down. The user only pushes the injection end at the front end of the syringe tube toward the injection portion. The syringe cylinder will retract toward the cover so that the syringe needle hidden in the syringe tube exposes out. Moreover, the user will not be hurt by the syringe needle when the user covers the cover.

### BACKGROUND OF THE INVENTION

The syringe discussed here is a medical hypodermic syringe which is widely used medically and thus people also take much attention to the security of the hypodermic syringe.

Moreover, syringes have needles which ate usually polluted by bacteria, virus, or impure blood, etc. If the syringes are not well processed before deserted, it is very possible that these syringes will become dangerous objects in the natural environment. Further, it is also possible that the medical persons are hurt by the needles. Thus medical persons are afraid to treat these medical wastes.

In the prior art, the syringe needles are covered by a hard cover for preventing from piercing others. For example, U.S. Pat. No. 5,490,810 discloses a syringe with a sheath at one side of the syringe needle for receiving the needle. In U.S. Pat. No. 5,667,496, a double layered cover is disclosed. Similar structures are disclosed in U.S. Pat. Nos. 5,775,498 and 5,084,027.

In U.S. Pat. No. 6,158,790, a tool for removing a syringe needle is disclosed. In U.S. Pat. No. 6,179,812, a syringe needle capable of reducing to the syringe cylinder is disclosed. In U.S. Pat. No. 6,171,284, a packaging structure for packaging a syringe needle is disclosed.

In above said prior arts, before injection, the cover for covering the syringe needle must be taken down manually and after injection, the cover is returned to cover the syringe needle manually so that the wasted syringe can be deserted. However, in these operations, the needles are exposed so that it is possible that persons is hurt by the syringe needle.

### SUMMARY OF THE INVENTION

Accordingly, the primary object of the present invention is to provide a safety syringe needle, wherein a protecting cover for covering a syringe needle is disclosed which can reduce the possibility that the syringe needle to hurt anybody.

A further object of the present invention is to provide a safety syringe needle, wherein in injection, the protection cover is unnecessary to be detached. As the syringe needle is not used, the needle is hidden in the cover. A safety pin is used to prevent the syringe needle from exposure out. In injection, it is only necessary to release the safety pin. After injection, as the syringe needle is pulled out from the injection portion, the syringe needle will hid into the protecting cover automatically. In the operation, the protection cover is unnecessary to be detached so that the user will not be hurt in operation.

To achieve above objects, the present invention provides a syringe needle protecting cover which comprises a cover, a syringe tube and an elastic element. The cover has an outlet capable of being connected to a front end of a syringe cylinder. The cover receives a syringe needle. A syringe tube is a hollow tube for hiding the syringe needle therein. The syringe tube is coupled to the outlet of the cover; the syringe tube being movable in the outlet. The syringe needle is shielded by the syringe tube. An exposed end of the syringe tube has an injection end. The elastic element is located along the axial direction of the syringe tube. Two ends of the elastic element resisting against the outlet of the cover and the injection end of the syringe tube, respectively. By the elastic force of the elastic element, the syringe tube is pushed to the position hiding the syringe needle.

The various objects and advantages of the present invention will be more readily understood from the following detailed description when read in conjunction with the appended drawing.

### BRIEF DESCRIPTION OF THE DRAWINGS

FIG. 1 is a schematic view showing the connection of the syringe needle protecting cover of the present invention and a front end of a syringe cylinder.

FIG. 2 shows the structure of the present invention.

FIG. 3 shows that in the present invention, the syringe needle is hidden in a syringe tube.

FIG. 4 is a schematic view showing that in using the syringe needle protecting cover of the present invention, the syringe tube exposes out.

FIG. 5 is a schematic view showing the package of the present invention.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

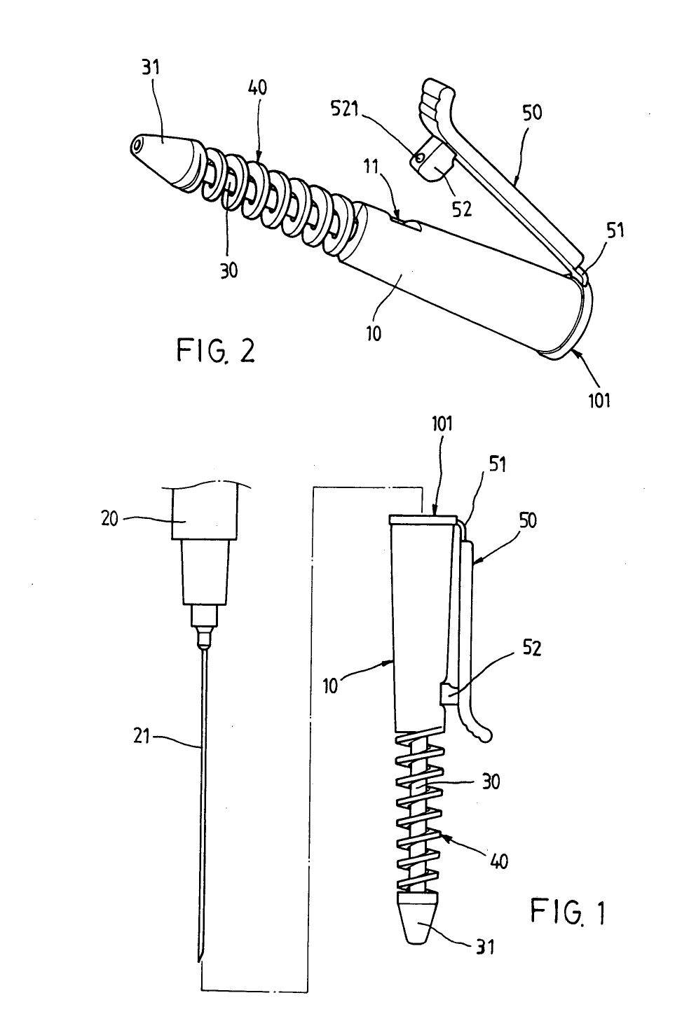

With reference to FIGS. 1 and 2, the syringe needle protecting cover of the present invention is illustrated. The syringe needle protecting cover comprises the following elements.

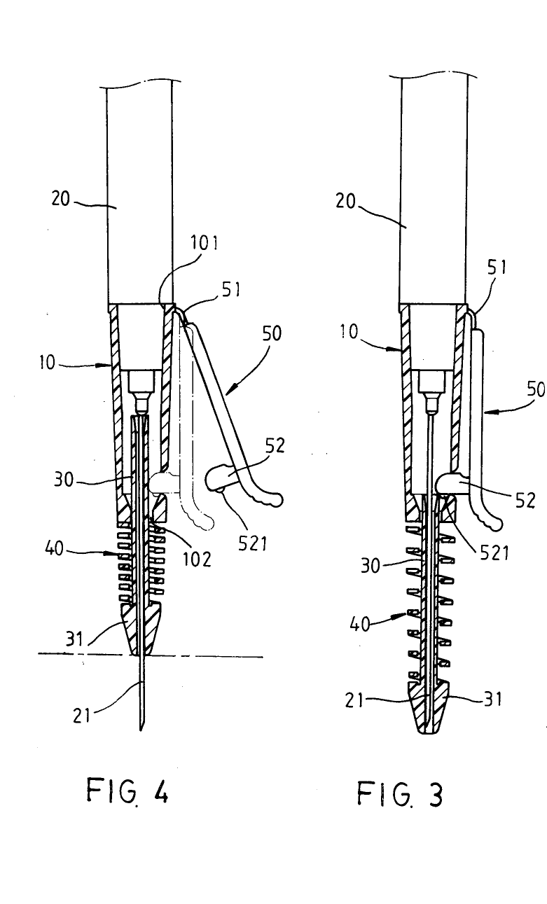

A cover 10 is a hollow tube which has an inlet 101 and an outlet 102 (see FIG. 4). The inlet 101 has a configuration capable of being connected to a front end of a syringe cylinder 20 (referring to FIG. 3). The cover 10 can receive a syringe needle 21.

A syringe tube 30 is a hollow tube for hiding the syringe needle 21 therein. The syringe tube 30 is coupled to the outlet 102 of the cover 10. The syringe tube 30 is movable in the outlet 102 along the axial direction of the cover 10. The syringe needle 21 is shielded by the syringe tube 30 (referring to FIG. 3). An exposed end of the syringe tube 30 has an injection end 31 which has a size larger than the outlet 102 of the cover 10. In syringe, the injection end 31 will contact the injection portion firstly. Then, the syringe tube 30 will be pushed by the injection portion so as to be reduced into the cover 10. Then the syringe needle 21 hidden in the syringe tube 30 will expose out from the front end of the syringe tube 30 (referring to FIG. 4).

An elastic element 40 is located along the axial direction of the syringe tube 30. One end of the elastic element 40 resists against the outlet 102 of the cover 10 and another end thereof resists against the injection end 31 of the syringe tube 30. By the elastic force of the elastic element 40, the syringe tube 30 is pushed to the position hiding the syringe needle 21 (referring to FIG. 3.).

Therefore, the syringe tube 30 of the present invention is telescopic and movable. In manufacturing, the syringe needle 21 of the syringe and the syringe needle protecting cover are installed at a front end of the syringe cylinder 20. By the ejection of the elastic element 40, the syringe needle 21 is hidden in the syringe tube 30 so that the syringe needle 21 will not expose to hurt people. When the syringe is used, the syringe needle protecting cover is unnecessary to be taken down. The user only pushes the injection end 31 at the front end of the syringe tube 30 toward the injection portion. The syringe tube 3.0 will retract toward the cover 10 so that the syringe needle 21 hidden in the syringe tube 30 exposes out. With the forward movement of the syringe cylinder 20, the syringe cylinder 20 pierces into the injection portion. After the operation, the syringe needle 21 is pulled out from the injection portion. The syringe tube 30 is pushed out from the cover 10 by the elastic element 40 so that the syringe needle 21 is further hidden in the syringe tube 30. In the process, the syringe needle protecting cover of the present invention is unnecessary to be detached. Thereby, the user will not be hurt by the syringe needle 21 when the user covers the cover.

In above embodiment, a safety pin 50 can be installed to the cover 10. One end of the safety pin 50 is connected to the cover 10 by a connecting portion 51. The connecting portion 51 is elastic and flexible. Another end of the connecting portion 51 has a coupling portion 52. The coupling portion 51 is embedded into a through hole 11 at one side of the cover 10. The coupling portion 51 has a tip 521 which can be buckled to the through hole 11 temporarily so as to prevent the releasing of the safety pin 50. The coupling portion 52 embedded into the through hole 11 has a sufficient embedding length and depth. Thereby, an edge of the coupling portiod 52 is placed at a moving path of the syringe tube 30 retracting into the cover 10. By the stop effect of the front edge of the coupling portion 52 (referring to FIG. 3), the syringe tube 30 will not retract due to a mistake action and thus the syringe needle 21 can expose out. Thereby, a safety effect is formed. Before injection, it is only necessary to release the stop effect of the safety pin 50 (referring to FIG. 4), the injection operation can be performed without needing to uncover the cover.

In realizing the embodiment of the present invention, the cover 10, syringe tube 30, elastic element 40, and safety pin 50 can be integrally formed by plastics so that the manufacturing process can be saved.

In this embodiment, the elastic element 40 can be a helical spring. It is preferable that the elastic element 40 winds around the syringe tube 30. Since the present invention can be made by plastics a preferred elasticity can be provided.

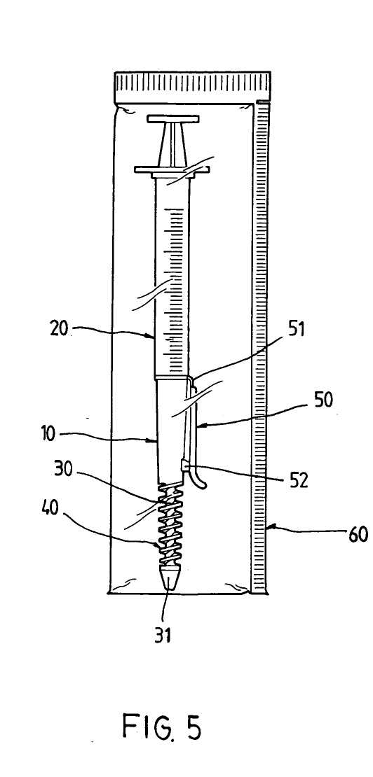

Referring to FIG. 5, when the syringe needle protecting cover is made, the syringe needle protecting cover can be packaged in a sterile sealing bag by a sterile way so that other objects will not pollute the syringe needle protecting cover.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A syringe needle protecting cover comprises:
a cover being a hollow tube which has an inlet and an outlet; the outlet has a configuration capable of being connected to a front end of a syringe cylinder; the cover receiving a syringe needle;
a syringe tube being a hollow tube for hiding the syringe needle therein; the syringe tube being coupled to the outlet of the cover; the syringe tube being movable in the outlet; along the axial direction of the cover; the syringe needle being shielded by the syringe tube; an exposed end of the syringe tube having an injection end which has a size larger than the outlet of the cover; in syringe, the injection end will contact the injection portion firstly; then, the syringe tube will be pushed by the injection portion so as to be reduced into the cover; then the syringe needle hidden in the syringe tube will expose out from the front end of the syringe tube;
an elastic element being located along the axial direction of the syringe tube; one end of the elastic element resisting against the outlet of the cover and another end thereof resisting against the injection end of the syringe tube; by the elastic force of the elastic element, the syringe tube being pushed to a position hiding the syringe needle.

2. The syringe needle protecting cover as claimed in claim 1, wherein the elastic element is a helical spring.

3. The syringe needle protecting cover as claimed in claim 1, wherein the elastic element is a helical spring wound around the syringe tube.

4. The syringe needle protecting cover as claimed in claim 1, wherein the syringe tube and the elastic element are integrally formed.

5. The syringe needle protecting cover as claimed in claim 1, wherein the syringe needle is packaged in a sealing bag.

6. The syringe needle protecting cover as claimed in claim 1, wherein a safety pin is installed to the cover; one end of the safety pin is connected to the cover by a connecting portion; the connecting portion is elastic and flexible; another end of the connecting portion has a coupling portion; the coupling portion is embedded into a through hole at one side of the cover; the coupling portion has a tip which can be buckled to the through hole temporarily so as to prevent the safety pin from releasing; the coupling portion embedded into the through hole has a sufficient embedding length and depth; an edge of the coupling portion is placed at a moving path of the syringe tube retracting into the cover; the syringe tube will not retract due to a mistake action and thus the syringe needle exposes out.

7. The syringe needle protecting cover as claimed in claim 6, wherein the coupling portion has a tip; the tip is buckled to the through hole so as to firmly secure the safety pin.

8. The syringe needle protecting cover as claimed in claim 6, wherein the cover, syringe tube, elastic element and safety pin are integrally formed.
